(19) United States
(12) Patent Application Publication
(10) Pub. No.: US 2024/0376030 A1
(43) Pub. Date: Nov. 14, 2024

(54) AZEOTROPE AND AZEOTROPE-LIKE COMPOSITIONS OF 1,1,2-TRIFLUOROETHANE (HFC-143) AND 1-CHLORO-1,1-DIFLUOROETHANE (HCFC-142B) AND APPLICATIONS THEREOF

(71) Applicant: Honeywell International Inc., Charlotte, NC (US)

(72) Inventors: Joshua Close, Cheektowaga, NY (US); Pramod K.W. Harikumar Warrier, Cheektowaga, NY (US); Justin Howard, Lakeview, NY (US); Terris Yang, East Amherst, NY (US); Haiyou Wang, Amherst, NY (US); Akbar Mahdavi-Shakib, Buffalo, NY (US)

(21) Appl. No.: 18/658,336

(22) Filed: May 8, 2024

Related U.S. Application Data
(60) Provisional application No. 63/465,502, filed on May 10, 2023.

(51) Int. Cl. C07C 17/25; C07C 17/383; C07C 19/08; C07C 19/12

(52) U.S. Cl. CPC C07C 17/25; C07C 17/383; C07C 19/08; C07C 19/12

(57) ABSTRACT

An azeotrope or azeotrope-like composition consisting essentially of effective amounts of 1-chloro-1,1-difluoroethane (HCFC-142b) and 1,1,2-trifluoroethane (HFC-143). Methods for separating the azeotrope or azeotrope-like composition and/or exploiting the composition in extractive and pressure swing distillation are also disclosed in connection with methods of manufacturing 1,1,2-trifluoroethane (HFC-143).

### CROSS-REFERENCE TO RELATED APPLICATIONS

This application claims the benefit under 35 U.S.C. § 119 (e) of U.S. Provisional Application No. 63/465,502 entitled “AZEOTROPE AND AZEOTROPE-LIKE COMPOSITIONS OF 1,1,2-TRIFLUOROETHANE (HFC-143) AND 1-CHLORO-1,1-DIFLUOROETHANE (HCFC-142B) AND APPLICATIONS THEREOF,” filed on May 10, 2023, which is herein incorporated by reference in its entirety.

### FIELD

The present disclosure relates to azeotrope and azeotrope-like compositions and, in particular, to azeotrope and azeotrope-like compositions consisting essentially of 1,1,2-trifluoroethane (HFC-143) and 1-chloro-1,1-difluoroethane (HCFC-142b) and applications or uses for these compositions.

### BACKGROUND

Fluorocarbon fluids have properties that are desirable for use as heat transfer media, immersion coolants, liquid or gaseous dielectrics, industrial refrigerants, and other applications.

For example, 1,2-difluoroethylene (HFO-1132) has recently found increased utility for a variety of uses. HFO-1132 may exist as a mixture of two geometric isomers, the E- or trans isomer and the Z- or cis isomer, which may be used separately or together in various proportions. Potential end use applications of HFO-1132 include refrigerants, either used alone or in blends with other components, solvents for organic materials, and as a chemical intermediate in the synthesis of other halogenated hydrocarbon solvents. Improved methods for the production of HFO-1132 and, in particular, HFO-1132E, are desired.

Azeotrope and azeotrope-like compositions may be encountered during the manufacture of fluorocarbon fluids and understanding any such azeotrope or azeotrope-like compositions is helpful to improve the efficiency of the manufacturing processes.

### SUMMARY

The present disclosure provides minimum-boiling, homogenous azeotrope or azeotrope-like compositions consisting essentially of 1-chloro-1,1-difluoroethane (HCFC-142b) and 1,1,2-trifluoroethane (HFC-143), and applications or uses for these compositions.

In one form thereof, the present disclosure provides an azeotrope or azeotrope-like composition consisting essentially of effective amounts of 1-chloro-1,1-difluoroethane (HCFC-142b) and 1,1,2-trifluoroethane (HFC-143).

In another form thereof, the present disclosure provides a method for producing 1,1,2-trifluoroethane (HFC-143) comprising hydrogenating 1-chloro-1,1-difluoroethane (HCFC-142b) with hydrogen (H₂) to form a product mixture comprising an azeotrope or azeotrope-like composition consisting essentially of effective amounts of 1-chloro-1,1-difluoroethane (HCFC-142b) and 1,1,2-trifluoroethane (HFC-143); and separating the 1-chloro-1,1-difluoroethane (HCFC-142b) and the 1,1,2-trifluoroethane (HFC-143) to provide a product composition comprising the 1,1,2-trifluoroethane (HFC-143). The separating may be performed by extractive or pressure swing distillation.

### BRIEF DESCRIPTION OF THE DRAWINGS

FIG. 1 is a process flow diagram of a process for separating 1-chloro-1,1-difluoroethane (HCFC-142b) and 1,1,2-trifluoroethane (HFC-143).

FIG. 2 is a graph of PTx measurements for an azeotrope or azeotrope-like composition of 1-chloro-1,1-difluoroethane (HCFC-142b) and 1,1,2-trifluoroethane (HFC-143) at an average temperature of 24.8° C.

FIG. 3 is a schematic of an apparatus for the separation by pressure swing distillation of an azeotrope or azeotrope-like composition of 1-chloro-1,1-difluoroethane (HCFC-142b) and 1,1,2-trifluoroethane (HFC-143).

FIG. 4 is a schematic of an apparatus for the separation by extractive distillation of an azeotrope or azeotrope-like composition of 1-chloro-1,1-difluoroethane (HCFC-142b) and 1,1,2-trifluoroethane (HFC-143).

### DETAILED DESCRIPTION

The present disclosure provides minimum-boiling, homogenous azeotropic or azeotrope-like compositions consisting essentially of 1-chloro-1,1-difluoroethane (HCFC-142b, also referred to herein as R142b) and 1,1,2-trifluoroethane (HFC-143, also referred to herein as R132) and applications or uses for these compositions.

### I. Description of Azeotrope or Azeotrope-Like Compositions

An “azeotrope” composition is a unique combination of two or more components. An azeotrope composition can be characterized in various ways. For example, at a given pressure, an azeotrope composition boils at a constant characteristic temperature which is either greater than the higher boiling point component (maximum boiling azeotrope) or less than the lower boiling point component (minimum boiling azeotrope). At this characteristic temperature the same composition will exist in both the vapor and liquid phases. The azeotrope composition does not fractionate upon boiling or evaporation. Therefore, the components of the azeotrope composition cannot be separated during a phase change.

An azeotrope composition is also characterized in that, at the characteristic azeotrope temperature, the bubble point pressure of the liquid phase is identical to the dew point pressure of the vapor phase.

The behavior of an azeotrope composition is in contrast with that of a non-azeotrope composition in which during boiling or evaporation, the liquid composition changes to a substantial degree.

For the purposes of the present disclosure, an azeotrope composition is characterized as that composition which boils at a constant characteristic temperature, the temperature being lower (a minimum boiling azeotrope) than the boiling points of the two or more components, and thereby having the same composition in both the vapor and liquid phases.

One of ordinary skill in the art would understand however that at different pressures, both the composition and the boiling point of the azeotrope composition will vary to some extent. Therefore, depending on the temperature and/or pressure, an azeotrope composition can have a variable composition. The skilled person would therefore understand that composition ranges, rather than fixed compositions, can be used to define azeotrope compositions. In addition, an azeotrope may be defined in terms of exact weight percentages of each component of the compositions characterized by a fixed boiling point at a specified pressure.

An “azeotrope-like” composition is a composition of two or more components which behaves substantially as an azeotrope composition. Thus, for the purposes of this disclosure, an azeotrope-like composition is a combination of two or more different components which, when in liquid form under given pressure, will boil at a substantially constant temperature, and which will provide a vapor composition substantially identical to the liquid composition undergoing boiling.

Azeotrope or azeotrope-like compositions can be identified using a number of different methods.

Static Vapor-Liquid Equilibrium Methods are a class of experimental techniques that can also be used to identify the presence of azeotrope and azeotrope-like compositions. One such technique, known as the PTx method, collects measurements of the total saturation pressure (“P”) exerted by mixtures of known compositions (“x”) at fixed temperatures (“T”) and cell volumes. (Walas, Phase Equilibria in Chemical Engineering, Butterworth-Heinemann, 1985, pp. 537). Using data collected from the PTx experiment, as well as pure component properties of constituents of the mixtures, the thermodynamic properties of the mixture can be accurately characterized by fitting the component's interaction parameters in a well-defined thermodynamic equation; one such equation is the Non-random, Two-Liquid (NRTL) activity coefficient model described by Renon and Prausnitz (Local Compositions in Thermodynamic Excess Functions for Liquid Mixtures, AlChE Journal, Vol. 14, January 1968, pp. 135-144).

The presence of an azeotrope and its corresponding composition can be observed by plotting saturation pressure measurements from PTx data and saturation pressures described by NRTL as a function of composition. For a given temperature (isotherm), the presence of an azeotrope composition is identified by the observation of a maximum or minimum in total pressure that is greater or less than the pure saturation pressures of any of the components alone.

As used herein, the term “consisting essentially of”, with respect to the components of an azeotrope or azeotrope-like composition or mixture, means the composition contains the indicated components in an azeotrope or azeotrope-like ratio, and may contain additional components provided that the additional components do not form new azeotrope or azeotrope-like systems. For example, azeotrope mixtures consisting essentially of two compounds are those that form binary azeotropes, which optionally may include one or more additional components, provided that the additional components do not render the mixture non-azeotropic and do not form an azeotrope with either or both of the compounds (e.g., do not form a ternary or higher azeotrope).

As used herein, the term “about”, when used in connection with recited weight percentages of the components of the present compositions, includes a deviation of ±0.3% from the recited weight percentage.

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

As previously discussed, for an azeotrope, at the maximum or minimum boiling point, the composition of the vapor phase will be identical to the composition of the liquid phase. The azeotrope-like composition is therefore that composition of components which provides a substantially constant minimum or maximum boiling point at which substantially constant boiling point the composition of the vapor phase will be substantially identical to the composition of the liquid phase.

### II. Azeotrope and Azeotrope-Like Compositions of 1-chloro-1,1-difluoroethane (HCFC-142b) and 1,1,2-trifluoroethane (HFC-143)

The present disclosure provides a minimum-boiling, homogenous azeotrope or azeotrope-like composition comprising effective amounts of 1-chloro-1,1-difluoroethane (HCFC-142b) and 1,1,2-trifluoroethane (HFC-143). The present disclosure particularly provides a minimum-boiling, homogenous azeotrope or azeotrope-like composition consisting essentially of effective amounts of 1-chloro-1,1-difluoroethane (HCFC-142b) and 1,1,2-trifluoroethane (HFC-143). The present disclosure provides a minimum-boiling, homogenous azeotrope or azeotrope-like composition consisting of effective amounts of 1-chloro-1,1-difluoroethane (HCFC-142b) and 1,1,2-trifluoroethane (HFC-143).

The azeotrope or azeotrope-like composition may comprise from about 6.2 wt. % to about 23.9 wt. % of 1,1,2-trifluoroethane (HFC-143) and from about 93.8 wt. % to about 76.1 wt. % of 1-chloro-1,1-difluoroethane (HCFC-142b) at a temperature of from about 0.1° C. to about 79.3° C. and a pressure of from about 21.1 psia to about 202.1 psia. The azeotrope or azeotrope-like composition may consist essentially of from about 6.2 wt. % to about 23.9 wt. % of 1,1,2-trifluoroethane (HFC-143) and from about 93.8 wt. % to about 76.1 wt. % of 1-chloro-1,1-difluoroethane (HCFC-142b) at a temperature of from about 0.1° C. to about 79.3° C. and a pressure of from about 21.1 psia to about 202.1 psia. The azeotrope or azeotrope-like composition may consist of from about 6.2 wt. % to about 23.9 wt. % of 1,1,2-trifluoroethane (HFC-143) and from about 93.8 wt. % to about 76.1 wt. % of 1-chloro-1,1-difluoroethane (HCFC-142b) at a temperature of from about 0.1° C. to about 79.3° C. and a pressure of from about 21.1 psia to about 202.1 psia.

The azeotrope or azeotrope-like compositions may comprise from about 0.1 wt. % to about 54.0 wt. % of 1,1,2-trifluoroethane (HFC-143) and from about 46.0 wt. % to about 99.9 wt. % of 1-chloro-1,1-difluoroethane (HCFC-142b), and more specifically, from about 7.0 wt. % to about 40.3 wt. % of 1,1,2-trifluoroethane (HFC-143) and from about 59.7 wt. % to about 93.0 wt. % of 1-chloro-1,1-difluoroethane (HCFC-142b), and more particularly, from about 15.6 wt. % to about 32.6 wt. % of 1,1,2-trifluoroethane (HFC-143) and from about 67.4 wt. % to about 84.4 wt. % of 1-chloro-1,1-difluoroethane (HCFC-142b), and still more specifically, about 23.9 wt. % of 1,1,2-trifluoroethane (HFC-143) and about 76.1 wt. % of 1-chloro-1,1-difluoroethane (HCFC-142b) at a pressure of about 202.1 psia.

The azeotrope or azeotrope-like compositions may consist essentially of from about 0.1 wt. % to about 54.0 wt. % of 1,1,2-trifluoroethane (HFC-143) and from about 46.0 wt. % to about 99.9 wt. % of 1-chloro-1,1-difluoroethane (HCFC-142b), and more specifically, from about 7.0 wt. % to about 40.3 wt. % of 1,1,2-trifluoroethane (HFC-143) and from about 59.7 wt. % to about 93.0 wt. % of 1-chloro-1,1-difluoroethane (HCFC-142b), and more particularly, from about 15.6 wt. % to about 32.6 wt. % of 1,1,2-trifluoroethane (HFC-143) and from about 67.4 wt. % to about 84.4 wt. % of 1-chloro-1,1-difluoroethane (HCFC-142b), and still more specifically, about 23.9 wt. % of 1,1,2-trifluoroethane (HFC-143) and about 76.1 wt. % of 1-chloro-1,1-difluoroethane (HCFC-142b) at a pressure of about 202.1 psia.

The azeotrope or azeotrope-like compositions may consist of from about 0.1 wt. % to about 54.0 wt. % of 1,1,2-trifluoroethane (HFC-143) and from about 46.0 wt. % to about 99.9 wt. % of 1-chloro-1,1-difluoroethane (HCFC-142b), and more specifically, from about 7.0 wt. % to about 40.3 wt. % of 1,1,2-trifluoroethane (HFC-143) and from about 59.7 wt. % to about 93.0 wt. % of 1-chloro-1,1-difluoroethane (HCFC-142b), and more particularly, from about 15.6 wt. % to about 32.6 wt. % of 1,1,2-trifluoroethane (HFC-143) and from about 67.4 wt. % to about 84.4 wt. % of 1-chloro-1,1-difluoroethane (HCFC-142b), and still more specifically, about 23.9 wt. % of 1,1,2-trifluoroethane (HFC-143) and about 76.1 wt. % of 1-chloro-1,1-difluoroethane (HCFC-142b) at a pressure of about 202.1 psia.

In other words, the compositions may comprise 1-chloro-1,1-difluoroethane (HCFC-142b) in an amount of as much as about 99.9 wt. %, or about 93.0 wt. %, or about 84.4 wt. %, or about 76.1 wt. %, or as little as about 46.0 wt. %, or about 59.7 wt. %, or about 67.4 wt. %, or by any two of the foregoing values as endpoints, such as from about 46.0 wt. % to about 99.9 wt. %, from about 59.7 wt. % to about 93.0 wt. %, from about 67.4 wt. % to about 84.4 wt. %, and/or about 76.1 wt. %, based on the total weight of the 1-chloro-1,1-difluoroethane (HCFC-142b) and 1,1,2-trifluoroethane (HFC-143) of the azeotrope or azeotrope-like composition at a pressure of about 202.1 psia.

In other words, the compositions may consist essentially of 1-chloro-1,1-difluoroethane (HCFC-142b) in an amount of as much as about 99.9 wt. %, or about 93.0 wt. %, or about 84.4 wt. %, or about 76.1 wt. %, or as little as about 46.0 wt. %, or about 59.7 wt. %, or about 67.4 wt. %, or by any two of the foregoing values as endpoints, such as from about 46.0 wt. % to about 99.9 wt. %, from about 59.7 wt. % to about 93.0 wt. %, from about 67.4 wt. % to about 84.4 wt. %, and/or about 76.1 wt. %, based on the total weight of the 1-chloro-1,1-difluoroethane (HCFC-142b) and 1,1,2-trifluoroethane (HFC-143) of the azeotrope or azeotrope-like composition at a pressure of about 202.1 psia.

In other words, the compositions may consist of 1-chloro-1,1-difluoroethane (HCFC-142b) in an amount of as much as about 99.9 wt. %, or about 93.0 wt. %, or about 84.4 wt. %, or about 76.1 wt. %, or as little as about 46.0 wt. %, or about 59.7 wt. %, or about 67.4 wt. %, or by any two of the foregoing values as endpoints, such as from about 46.0 wt. % to about 99.9 wt. %, from about 59.7 wt. % to about 93.0 wt. %, from about 67.4 wt. % to about 84.4 wt. %, and/or about 76.1 wt. %, based on the total weight of the 1-chloro-1,1-difluoroethane (HCFC-142b) and 1,1,2-trifluoroethane (HFC-143) of the azeotrope or azeotrope-like composition at a pressure of about 202.1 psia.

In other words, the compositions may comprise 1,1,2-trifluoroethane (HFC-143) in an amount as much as about 54.0 wt. %, or about 40.3 wt. %, or about 32.6 wt. %, or about 23.9 wt. %, or as little as about 0.1 wt. %, or about 7.0 wt. %, or about 15.6 wt. %, or by any two of the foregoing values as endpoints, such as from about 0.1 wt. % to about 54.0 wt. %, from about 7.0 wt. % to about 40.3 wt. %, from about 15.6 wt. % to about 32.6 wt. %, and/or about 23.9 wt. %, based on the total weight of the 1-chloro-1,1-difluoroethane (HCFC-142b) and 1,1,2-trifluoroethane (HFC-143) of the azeotrope or azeotrope-like composition at a pressure of about 202.1 psia.

In other words, the compositions may consist essentially of 1,1,2-trifluoroethane (HFC-143) in an amount as much as about 54.0 wt. %, or about 40.3 wt. %, or about 32.6 wt. %, or about 23.9 wt. %, or as little as about 0.1 wt. %, or about 7.0 wt. %, or about 15.6 wt. %, or by any two of the foregoing values as endpoints, such as from about 0.1 wt. % to about 54.0 wt. %, from about 7.0 wt. % to about 40.3 wt. %, from about 15.6 wt. % to about 32.6 wt. %, and/or about 23.9 wt. %, based on the total weight of the 1-chloro-1,1-difluoroethane (HCFC-142b) and 1,1,2-trifluoroethane (HFC-143) of the azeotrope or azeotrope-like composition at a pressure of about 202.1 psia.

In other words, the compositions may consist of 1,1,2-trifluoroethane (HFC-143) in an amount as much as about 54.0 wt. %, or about 40.3 wt. %, or about 32.6 wt. %, or about 23.9 wt. %, or as little as about 0.1 wt. %, or about 7.0 wt. %, or about 15.6 wt. %, or by any two of the foregoing values as endpoints, such as from about 0.1 wt. % to about 54.0 wt. %, from about 7.0 wt. % to about 40.3 wt. %, from about 15.6 wt. % to about 32.6 wt. %, and/or about 23.9 wt. %, based on the total weight of the 1-chloro-1,1-difluoroethane (HCFC-142b) and 1,1,2-trifluoroethane (HFC-143) of the azeotrope or azeotrope-like composition at a pressure of about 202.1 psia.

The compositions may have azeotropic or azeotrope-like characteristics at a temperature of about 0.1° C., about 9.8° C., about 19.8° C., about 24.8° C., about 29.7° C., about 34.7° C., about 39.6° C., about 44.6° C., about 49.5° C., about 54.6° C., about 59.5° C., about 64.4° C., about 69.3° C., about 74.3° C., and/or about 79.3° C., or within any range encompassed by any two of the foregoing values as endpoints, such as from about 0.1° C. to about 79.3° C.

The compositions may have azeotropic or azeotrope-like characteristics at a pressure of about 21.1 psia, about 30.0 psia, about 41.9 psia, about 49.0 psia, about 57.0 psia, about 66.0 psia, about 75.9 psia, about 87.1 psia, about 99.2 psia, about 113.2 psia, about 127.8 psia, about 143.7 psia, about 161.4 psia, about 180.9 psia, about 202.1 psia, or within any range encompassed by any two of the foregoing values as endpoints, such as from about 21.1 psia to about 202.1 psia.

Specifically, and as described in Table 1 below, the azeotrope or azeotrope-like composition of 1-chloro-1,1-difluoroethane (HCFC-142b) and 1,1,2-trifluoroethane (HFC-143) may correlate with pressure (psia) and saturation temperature.

In column (i) of Table 1 below, temperature glide is the difference between the saturated vapor temperature and the saturated liquid temperature at a fixed pressure in thermodynamic equilibrium. Consequently, an azeotrope composition may have a temperature glide of zero and an azeotrope-like composition has a temperature glide that is substantially close to zero. It has been identified that a temperature glide less than 0.5° C. is substantially close to zero and therefore compositions that satisfy such temperature glide are considered azeotrope-like. This method was used to determine the relative compositions in column (i) of Table 1 below, which may be regarded as the broadest azeotrope-like composition range.

In column (ii) of Table 1 below, the relative volatility is the ratio of the vapor composition to the liquid composition of the most volatile component relative to the ratio of the vapor composition to the liquid composition of the less volatile component at a fixed pressure in thermodynamic equilibrium. Consequently, an azeotrope composition has a relative volatility of 1.0 and an azeotrope-like composition has a relative volatility that is substantially close to 1.0. It has been identified that a relative volatility of 1.1 is substantially close to 1.0 and therefore compositions that satisfy such relative volatility are considered azeotrope-like. This method was used to determine the relative compositions in column (ii) of Table 1 below, which may be regarded as an intermediate azeotrope-like composition range.

In column (iii) of Table 1 below, a relative volatility of 1.05 is substantially close to 1.0 and therefore, compositions that satisfy such relative volatility are considered azeotrope-like. This may be regarded as the narrowest azeotrope-like composition range.

Column (1) of Table 1 below describes the azeotrope composition that, at a given pressure, the azeotrope composition boils at a constant characteristic temperature less than the lower boiling point component (minimum boiling azeotrope). At this characteristic temperature the same composition will exist in both the vapor and liquid phases. Therefore, the components of the azeotrope composition cannot be separated during a phase change and are regarded as the azeotrope composition. Such compositional values are presented in Column (1) of Table 1 below corresponding to each pressure and saturation temperature.

Accordingly, and in view of the foregoing, the azeotrope or azeotrope-like composition of 1-chloro-1,1-difluoroethane (HCFC-142b) and 1,1,2-trifluoroethane (HFC-143) may comprise any of the values as described in each of columns (1), (i), (ii) or (iii) in each row of Table 1 below.

In another embodiment, the azeotrope or azeotrope-like composition of 1-chloro-1,1-difluoroethane (HCFC-142b) and 1,1,2-trifluoroethane (HFC-143) may consist essentially of any of the values as described in each of columns (1), (i), (ii) or (iii) in each row of Table 1 below.

In a further embodiment, the azeotrope or azeotrope-like composition of 1-chloro-1,1-difluoroethane (HCFC-142b) and 1,1,2-trifluoroethane (HFC-143) may consist of any of the values as described in each of columns (1), (i), (ii) or (iii) in each row of Table 1 below.

TABLE 1
1) Azeotrope Composition and (2) Azeotrope-like composition ranges of 1-
chloro-1,1-difluoroethane (HCFC-142b) and 1,1,2-trifluoroethane (HFC-143)

| | | | | (2) Azeotropic-Like Composition Range | | | | | |
|---|---|---|---|---|---|---|---|---|---|
| | | (1) Azeotropic | | (i) | | (ii) | | (iii) | |
| | | Composition | | Glide <0.5° C. | | Rel. Vol <1.1 | | Rel. Vol <1.05 | |
| | Saturation | Wt. % | Wt. % | Wt. % | Wt. % | Wt. % | Wt. % | Wt. % | Wt. % |
| Pressure | Temp. | HFC- | HCFC- | HFC- | HCFC- | HCFC- | HFC- | HFC- | HCFC- |
| (psia) | (° C.) | 143 | 142b | 143 | 142(b) | 143 | 142(b) | 143 | 142(b) |
| 21.1 | 0.1 | 6.2 | 93.8 | 0.1 to | 71.5 to | 2.2 to | 81.4 to | 6.0 to | 85.3 to |
| | | | | 28.5 | 99.9 | 18.6 | 97.8 | 14.7 | 94.0 |
| 30.0 | 9.8 | 8.6 | 91.4 | 0.1 to | 68.9 to | 3.6 to | 78.8 to | 8.0 to | 82.9 to |
| | | | | 31.1 | 99.9 | 21.2 | 96.4 | 17.1 | 92.0 |
| 41.9 | 19.8 | 11.0 | 89.0 | 0.1 to | 66.1 to | 4.8 to | 76.2 to | 9.6 to | 80.7 to |
| | | | | 33.9 | 99.9 | 23.8 | 95.2 | 19.3 | 90.4 |
| 49.0 | 24.8 | 12.2 | 87.8 | 0.1 to | 64.7 to | 5.3 to | 74.9 to | 10.3 to | 79.6 to |
| | | | | 35.3 | 99.9 | 25.1 | 94.7 | 20.4 | 89.7 |
| 57.0 | 29.7 | 13.4 | 86.6 | 0.1 to | 63.3 to | 5.8 to | 73.6 to | 11.0 to | 78.5 to |
| | | | | 36.7 | 99.9 | 26.4 | 94.2 | 21.5 | 89.0 |
| 66.0 | 34.7 | 14.5 | 85.5 | 0.1 to | 61.8 to | 6.2 to | 72.4 to | 11.7 to | 77.4 to |
| | | | | 38.2 | 99.9 | 27.6 | 93.8 | 22.6 | 88.3 |
| 75.9 | 39.6 | 15.7 | 84.3 | 0.1 to | 60.1 to | 6.6 to | 71.1 to | 12.3 to | 76.3 to |
| | | | | 39.8 | 99.9 | 28.9 | 93.4 | 23.7 | 87.7 |
| 87.1 | 44.6 | 16.8 | 83.2 | 0.1 to | 58.7 to | 6.9 to | 69.8 to | 12.8 to | 75.3 to |
| | | | | 41.3 | 99.9 | 30.2 | 93.1 | 24.7 | 87.2 |
| 99.2 | 49.5 | 17.9 | 82.1 | 0.1 to | 57.1 to | 7.1 to | 68.5 to | 13.3 to | 74.2 to |
| | | | | 42.9 | 99.9 | 31.5 | 92.9 | 25.8 | 86.7 |
| 113.2 | 54.6 | 19.0 | 81.0 | 0.1 to | 55.3 to | 7.3 to | 67.1 to | 13.9 to | 73.1 to |
| | | | | 44.7 | 99.9 | 32.9 | 92.7 | 26.9 | 86.1 |
| 127.8 | 59.5 | 20.1 | 79.9 | 0.1 to | 53.7 to | 7.4 to | 65.7 to | 14.3 to | 72.0 to |
| | | | | 46.3 | 99.9 | 34.3 | 92.6 | 28.0 | 85.7 |
| 143.7 | 64.4 | 21.1 | 78.9 | 0.1 to | 51.9 to | 7.5 to | 64.3 to | 14.7 to | 70.9 to |
| | | | | 48.1 | 99.9 | 35.7 | 92.5 | 29.1 | 85.3 |
| 161.4 | 69.3 | 22.0 | 78.0 | 0.1 to | 50.1 to | 7.4 to | 62.8 to | 15.0 to | 69.8 to |
| | | | | 49.9 | 99.9 | 37.2 | 92.6 | 30.2 | 85.0 |
| 180.9 | 74.3 | 23.0 | 77.0 | 0.1 to | 48.1 to | 7.3 to | 61.3 to | 15.4 to | 68.6 to |
| | | | | 51.9 | 99.9 | 38.7 | 92.7 | 31.4 | 84.6 |
| 202.1 | 79.3 | 23.9 | 76.1 | 0.1 to | 46.0 to | 7.0 to | 59.7 to | 15.6 to | 67.4 to |
| | | | | 54.0 | 99.9 | 40.3 | 93.0 | 32.6 | 84.4 |

### III. Production of E-1,2-difluoroethylene (HFO-1132E)

It has been found that azeotrope or azeotrope-like compositions of 1-chloro-1,1-difluoroethane (HCFC-142b) and 1,1,2-trifluoroethane (HFC-143) may be formed or otherwise encountered during production of E-1,2-difluoroethylene (HFO-1132E).

In particular, azeotrope or azeotrope-like compositions of 1-chloro-1,1-difluoroethane (HCFC-142b) and 1,1,2-trifluoroethane (HFC-143) may be formed or otherwise encountered in a method for producing E-1,2-difluoroethylene (HFO-1132E) from 1-chloro-1,1-difluoroethane (HCFC-142b) according to a three-step scheme or process shown below (“Scheme 1”).

Scheme 1 includes the following three steps: (i) hydrogenating 1,1,2-trichloro-1,2,2-trifluoroethane (CFC-113) to produce 1,1,2-trifluoroethane (HFC-143), (ii) dehydrofluorinating 1,1,2-trifluoroethane (HFC-143) to produce a mixture of trans-1,2-difluoroethylene (HFO-1132E) and cis-1,2-difluoroethylene (HFO-1132Z), and (iii) isomerizing cis-1,2-difluoroethylene (HFO-1132Z) to trans-1,2-difluoroethylene (HFO-1132E).

Schematic equations for the three steps of Scheme 1 are represented below:

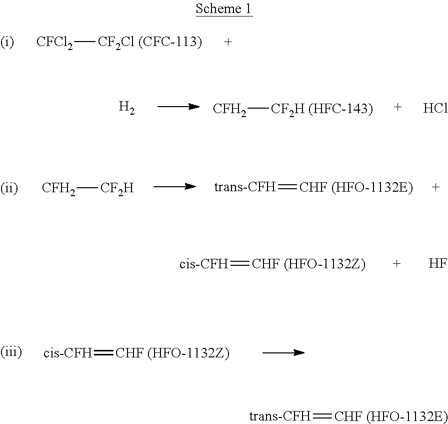

Step (i) may proceed through an intermediate of 1,1,2-trifluoroethene (HFC-143), wherein 1,1,2-trichloro-1,2,2-trifluoroethane (CFC-113) is first hydrogenated to produce the 1,1,2-trifluoroethene (HFC-143) as an intermediate, which intermediate is itself then hydrogenated to produce 1,1,2-trifluoroethane (HFC-143).

The azeotrope or azeotrope-like compositions of 1-chloro-1,1-difluoroethane (HCFC-142b) and 1,1,2-trifluoroethane (HFC-143) are formed in step (i) of Scheme 1 above. Here, the 1-chloro-1,1-difluoroethane (HCFC-142b) may be deemed an undesirable byproduct, where separation and subsequent removal of the 1-chloro-1,1-difluoroethane (HCFC-142b) byproduct may enhance the overall recovery of the 1,1,2-trifluoroethane (HFC-143) product. Therefore, it may be important to use or exploit such azeotrope or azeotrope-like compositions of 1-chloro-1,1-difluoroethane (HCFC-142b) and 1,1,2-trifluoroethane (HFC-143) to enhance the operation of Scheme 1 to produce E-1,2-difluoroethylene (HFO-1132E) in desired amounts or purities. For example, by separating the azeotrope or azeotrope-like compositions of 1-chloro-1,1-difluoroethane (HCFC-142b) and 1,1,2-trifluoroethane (HFC-143), the recovery purity of 1,1,2-trifluoroethane (HFC-143) is enhanced by substantially eliminating the 1-chloro-1,1-difluoroethane (HCFC-142b) from the 1,1,2-trifluoroethane (HFC-143).

The present disclosure additionally relates to a method for producing 1,1,2-trifluoroethane (HFC-143) comprising: hydrogenating 1,1,2-trichloro-1,2,2-trifluoroethane (CFC-113) with hydrogen (H2) to form a product mixture, the product mixture comprising an azeotrope or azeotrope-like composition consisting essentially of effective amounts of 1-chloro-1,1-difluoroethane (HCFC-142b) and 1,1,2-trifluoroethane (HFC-143); and separating the 1-chloro-1,1-difluoroethane (HCFC-142b) and the 1,1,2-trifluoroethane (HFC-143) to provide a product composition comprising the 1,1,2-trifluoroethane (HFC-143). The azeotrope or azeotrope-like composition provided in this method consists essentially of effective amounts of 1-chloro-1,1-difluoroethane (HCFC-142b) and 1,1,2-trifluoroethane (HFC-143) as defined in the disclosure above.

The separating step may comprise conveying the product mixture to a first column having a first pressure; collecting a first bottoms product from the first column; conveying a first distillate from the first column to a second column having a second pressure to provide a second distillate and a second bottoms product, the second distillate comprising the azeotrope or azeotrope-like composition; and collecting a second bottoms product from the second column. The first column pressure may be lower than the second column pressure, where the first bottoms product consists essentially of 1,1,2-trifluoroethane (HFC-143) and the second bottoms product consists essentially of 1-chloro-1,1-difluoroethane (HCFC-142b). Alternatively, the first column pressure may be higher than the second column pressure, where the first bottoms product consists essentially of 1-chloro-1,1-difluoroethane (HCFC-142b) and the second bottoms product consists essentially of 1,1,2-trifluoroethane (HFC-143). The separation step may comprise the additional step of recycling the second distillate to the first column.

Alternatively, the separating step may comprise conveying the product mixture and an entrainer fluid to a first column; collecting a first distillate from the first column comprising a first component of the azeotrope or azeotrope-like composition; collecting a first bottoms product from the first column comprising a mixture of the entrainer and a second component of the azeotrope or azeotrope-like composition; conveying the first bottoms product to a second column to separate the entrainer and the second component of the azeotrope or azeotrope-like composition; and removing a composition consisting essentially of the second component of the azeotrope or azeotrope-like composition as a second distillate from the second column. The first component of the azeotrope or azeotrope-like composition may consist of 1-chloro-1,1-difluoroethane (HCFC-142b), and the second component of the azeotrope or azeotrope-like composition consists of 1,1,2-trifluoroethane (HFC-143). Alternatively, the first component of the azeotrope or azeotrope-like composition may consist of 1,1,2-trifluoroethane (HFC-143), and the second component of the azeotrope or azeotrope-like composition consists of 1-chloro-1,1-difluoroethane (HCFC-142b). The separation step may comprise the additional step of recycling a second bottoms product consisting essentially of the entrainer from the second column to the first column.

It will be appreciated that step (i) of Scheme 1 as disclosed above in relation to the production of HFO-1132E discloses a method of producing HFC-143. Therefore, all features disclosed in relation to step (i) of Scheme 1 below, also apply to the method of producing HFC-143.

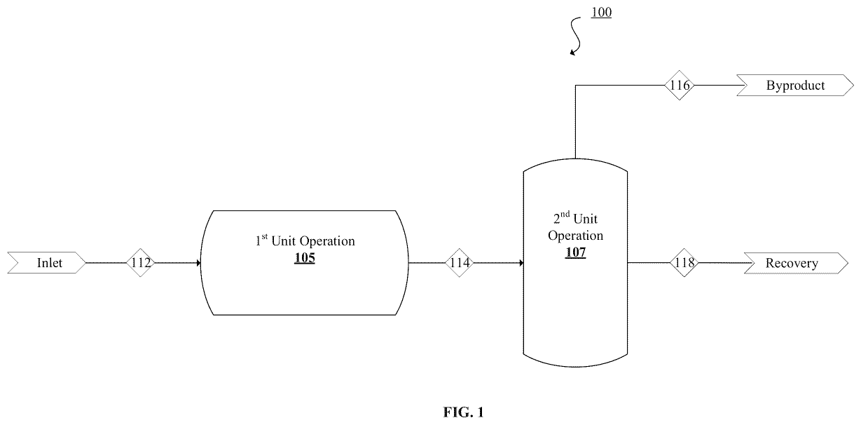

For example, and as illustrated in FIG. 1, an inlet stream 110 to process 100 comprising at least the reactants of Step (i): CFCl2-CF2Cl (CFC-113)+H2, may be mixed and sent to first unit operation 105.

First unit operation 105 may be a hydrogenation reactor whereas Step (i) of Scheme 1 is performed. Here, first unit operation 105 may be a tubular reactor made from a material which is resistant to temperature and/or corrosion such as nickel and its alloys, including Hastelloy (for example, Hastelloy C276), Inconel (for example, Inconel 600), Incoloy, and Monel, and the vessels may be lined with fluoropolymers. The hydrogenation reaction of Step (i) may be carried out in the gas or vapor phase, where the reactor may be first cleaned and flushed with an inert gas such as nitrogen, followed by packing with a catalyst. The catalyst may comprise a metal such as palladium, platinum, rhodium, ruthenium, iron, cobalt or nickel. More particularly, the catalyst may comprise a palladium metal, platinum metal, or a combination of palladium metal and platinum metal. The catalyst may be supported on a suitable support, such as carbon or alumina. For instance, the catalyst may be palladium on a carbon support, may be platinum on a carbon support, and/or may be palladium or platinum on an alumina support.

Reactant stream 112 flows through a bed of the catalyst (e.g., in either the up or down direction) within first unit operation 105, undergoing the hydrogenation reaction of Step (i) of Scheme 1. Here, the reaction temperature may be as low as about 100° C., about 125° C., about 150° C., about 200° C., about 250° C. or as high as about 300° C., about 350° C., about 400° C., or within any range encompassed by two of the foregoing values as endpoints, such as from about 100° C. to about 250° C., or from about 150° C. to about 200° C., for example. The temperature may be preferably from about 100° C. to about 350° C., and more preferably from about 200° C. to about 300° C. The contact time of the reactants with the catalyst may be as little as about 0.1 second, about 1 second, about 5 seconds, about 10 seconds, about 15 seconds or about 20 seconds, or as long as about 25 seconds, about 30 seconds, about 40 seconds, about 50 seconds, about 60 seconds, about 120 seconds, about or within any range encompassed by two of the foregoing values as endpoints. For example, the contact time may be preferably from about 1 second to about 120 seconds. The pressure may be as little as about 1 psig, about 3 psig, about 5 psig, about 10 psig, about 15 psig, about 20 psig, about 30 psig, about 35 psig or about 40 psig, or as great as about 90 psig, about 100 psig, about 120 psig, about 150 psig, about 200 psig or about 250 psig, about 300 psig, or within any range encompassed by two of the foregoing values as endpoints. For example, the pressure may be preferably from about 10 psig to about 300 psig.

Once reacted, reactant stream 112 forms product stream 114 which includes any one of, or combination of HCl, 1,1,2-trichloro-1,2,2-trifluoroethane (CFC-113), 1,1,2-trifluoroethane (HFC-143), and the azeotrope or azeotrope-like composition of 1-chloro-1,1-difluoroethane (HCFC-142b) and 1,1,2-trifluoroethane (HFC-143). As described previously, 1-chloro-1,1-difluoroethane (HCFC-142b) may be deemed an undesirable byproduct which is preferred to remove as a byproduct. Therefore, the separation of 1-chloro-1,1-difluoroethane (HCFC-142b) from 1,1,2-trifluoroethane (HFC-143), and subsequent removal of 1-chloro-1,1-difluoroethane (HCFC-142b) may be desirable to enhance the recovery of 1,1,2-trifluoroethane (HFC-143), leading to the enhanced production of E-1,2-difluoroethylene (HFO-1132E).

Specifically, once exiting first unit operation 105, product stream 114 may enter second unit operation 107. Second unit operation 107 may separate the components of the azeotrope or azeotrope-like compositions of 1-chloro-1,1-difluoroethane (HCFC-142b) and 1,1,2-trifluoroethane (HFC-143) from one another, such as by pressure swing distillation, extractive distillation, pervaporation, adsorption such as pressure swing adsorption, membrane separation, and the like. Pressure swing distillation is specifically described in further detail in Section IV, and extractive distillation is specifically described in further detail in Section V, both provided herein.

Once separated, the HCl and the 1,1,2-trifluoroethane (HFC-143) are recovered in recovery stream 118, and the undesirable byproduct 1-chloro-1,1-difluoroethane (HCFC-142b) may be removed in byproduct stream 116. Specifically, the amount or purity of 1,1,2-trifluoroethane (HFC-143) in recovery stream 118 may be greater than 90 mol %, 95 mol %, 97 mol %, greater than 99 mol %, or greater than 99.5 mol %, for example, based on total moles of organic components in the composition. The amount of 1-chloro-1,1-difluoroethane (HCFC-142b) in recovery stream 118 may be less than 5000 ppm, 3000 ppm, 2000 ppm, less than 1000 ppm, less than 500 ppm, or less than 250 ppm, for example, based on total moles of organic components in the composition.

Although unillustrated, recovery stream 118 may be subsequently sent to a further unit operation to separate the HCl from the 1,1,2-trifluoroethane (HFC-143), where the unit operation may be any one of, or combination of, an absorption unit, an adsorption unit, a membrane separator, a cryogenic separator, a subsequent chemical reactor, a distillation unit, and the like. Once the HCl is removed from the 1,1,2-trifluoroethane (HFC-143), the 1,1,2-trifluoroethane (HFC-143) may be utilized as the reactant in Step (ii).

### IV. Separating Azeotrope and Azeotrope-Like Compositions of 1-chloro-1,1-difluoroethane (HCFC-142b) and 1,1,2-trifluoroethane (HFC-143) via Pressure Swing Distillation

The present disclosure provides a method for separating an azeotrope or azeotrope-like composition of 1-chloro-1,1-difluoroethane (HCFC-142b) and 1,1,2-trifluoroethane (HFC-143) via pressure swing distillation, as discussed below and in Example 3.

In a first step, a mixture of 1-chloro-1,1-difluoroethane (HCFC-142b) and 1,1,2-trifluoroethane (HFC-143) is conveyed to a first, low-pressure column having a first pressure to provide a first distillate and a first bottoms product. The first bottoms product is an enriched stream of 1,1,2-trifluoroethane (HFC-143) which is collected from the bottom of the low-pressure column. The first distillate is an azeotrope or azeotrope-like composition of 1-chloro-1,1-difluoroethane (HCFC-142b) and 1,1,2-trifluoroethane (HFC-143) and is collected from the top of the low-pressure column and conveyed to a second, high-pressure column to provide a second distillate from the top of the high-pressure column and a second bottoms product from the bottom of the high-pressure column. The second bottoms product is an enriched steam of 1-chloro-1,1-difluoroethane (HCFC-142b) and is collected from the high-pressure column. The second distillate includes an azeotrope or azeotrope-like composition consisting essentially of effective amounts of 1-chloro-1,1-difluoroethane (HCFC-142b) and 1,1,2-trifluoroethane (HFC-143), which may be recycled back to the low-pressure column.

The above process may be modified to reverse the sequence of the low and high-pressure columns, wherein the high-pressure column is the first column and the low-pressure column is the second column and, when so modified, the first bottoms product is an enriched stream of 1-chloro-1,1-difluoroethane (HCFC-142b) and the second bottoms stream is an enriched stream of 1,1,2-trifluoroethane (HFC-143).

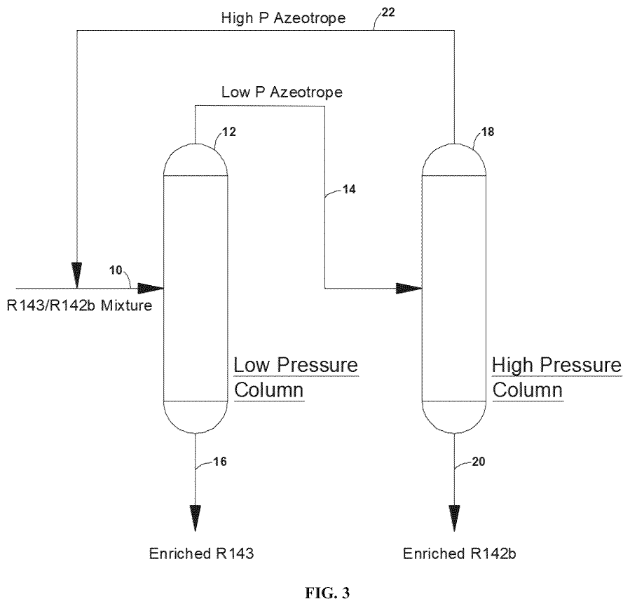

A schematic of an exemplary separation apparatus is provided in FIG. 3. Referring to this figure, a mixture of 1-chloro-1,1-difluoroethane (HCFC-142b) and 1,1,2-trifluoroethane (HFC-143) is fed into a first low-pressure column 12 as feed stream 10. The low-pressure column 12 provides a first distillate stream 14 which is an azeotrope or azeotrope-like composition of 1-chloro-1,1-difluoroethane (HCFC-142b) and 1,1,2-trifluoroethane (HFC-143) and a first bottoms product 16 which may be enriched in, or consist essentially of, HFC-143. The first distillate stream 14 is then conveyed to a high-pressure column 18 to provide a second distillate stream 22 which is an azeotrope or azeotrope-like composition of 1-chloro-1,1-difluoroethane (HCFC-142b) and 1,1,2-trifluoroethane (HFC-143) and a second bottoms product 20. The second bottoms product 20 is enriched in, or consists essentially of, HCFC-142B. The second distillate stream 22 may optionally be fed back into feed stream 10 and thereby recycled into the low-pressure column 12.

According to this method, if an azeotrope or azeotrope-like composition of 1-chloro-1,1-difluoroethane (HCFC-142b) and 1,1,2-trifluoroethane (HFC-143) is formed during the production of E-1,2-dichoroethane (HFO-1132E), for example according to Scheme 1, such as during step (i) of Scheme 1 during the production of 1,1,2-trifluoroethane (HFC-143), the azeotrope or azeotrope-like composition of 1-chloro-1,1-difluoroethane (HCFC-142b) and 1,1,2-trifluoroethane (HFC-143) may be separated into its constituent 1-chloro-1,1-difluoroethane (HCFC-142b) and 1,1,2-trifluoroethane (HFC-143) components, which may each then either be removed or recycled back into a suitable location in the production process.

### V. Separating Azeotrope and Azeotrope-Like Compositions of 1-chloro-1,1-difluoroethane (HCFC-142b) and 1,1,2-trifluoroethane (HFC-143) via Extractive Distillation

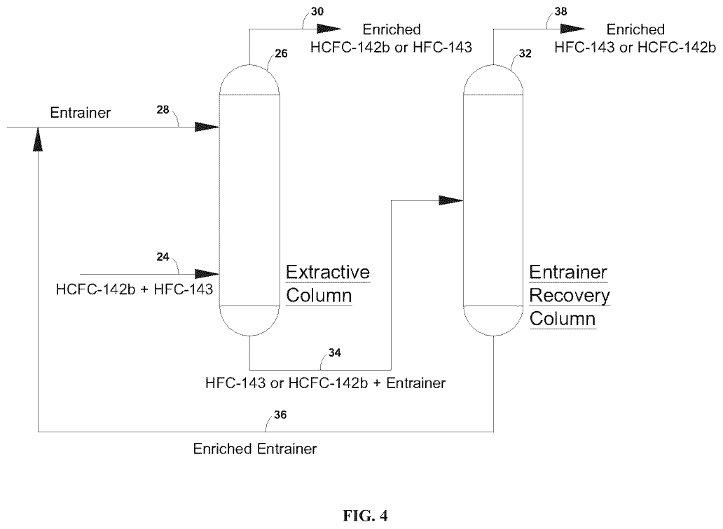

The present disclosure provides a method for separating an azeotrope or azeotrope-like composition of 1-chloro-1,1-difluoroethane (HCFC-142b) and 1,1,2-trifluoroethane (HFC-143) via extractive distillation, as discussed below and in Example 4. A schematic of an exemplary separation apparatus is provided in FIG. 4.

In a first step, a product stream 24 (which may be the same as product stream 114 with reference to FIG. 1), which includes at least the azeotrope or azeotrope-like composition of 1-chloro-1,1-difluoroethane (HCFC-142b) and 1,1,2-trifluoroethane (HFC-143) is conveyed to extractive column 26 along with an entrainer fluid 28. The entrainer fluid 28 is a composition where one of the components of the azeotrope or azeotrope-like composition of 1-chloro-1,1-difluoroethane (HCFC-142b) and 1,1,2-trifluoroethane (HFC-143) has a higher affinity for the entrainer fluid, as compared to the other. Here, one of the components of the azeotrope or azeotrope-like composition dissolves in the entrainer fluid 28 more readily than the other component. The entrainer fluid 28 therefore may be used to “break” the azeotrope or azeotrope-like composition into individual components, based upon the affinity/solubility difference, and therefore, utilized to selectively separate each of the components of the azeotrope or azeotrope-like composition. For example, the entrainer fluid 28 may have a higher affinity for 1-chloro-1,1-difluoroethane (HCFC-142b) and may act as a selective solute for the 1-chloro-1,1-difluoroethane (HCFC-142b) of the azeotrope or azeotrope-like composition. In another example, the entrainer fluid 28 may have a higher affinity for the 1,1,2-trifluoroethane (HFC-143) and may act as a selective solute for the 1,1,2-trifluoroethane (HFC-143) of the azeotrope or azeotrope-like composition.

Extractive column 26 is operated such that the operational parameters (temperature and pressure) separate the mixture of the entrainer fluid 28 and the first/dissolved component of the azeotrope or azeotrope-like composition from the second component of the azeotrope or azeotrope-like composition. For example, in the case where the entrainer fluid 28 has a higher affinity for 1-chloro-1,1-difluoroethane (HCFC-142b) and the entrainer fluid 28 are recovered in bottoms product 34 from extractive column 26, while enriched 1,1,2-trifluoroethane (HFC-143) is recovered as the distillate 30. In another example, in the case where the entrainer fluid 28 has a higher affinity for 1,1,2-trifluoroethane (HFC-143), 1,1,2-trifluoroethane (HFC-143) and the entrainer fluid 28 are recovered in bottoms product 34, 1-chloro-1,1-difluoroethane (HCFC-142b) is recovered as the distillate 30.

In a second step, the bottoms product 34 containing each of the mixture of the entrainer fluid and the dissolved first component of the azeotrope or azeotrope-like composition is conveyed to recovery column 32. Recovery column 32 is operated such that the operational parameters (temperature and pressure) separate the entrainer fluid and the dissolved component of the azeotrope or azeotrope-like composition from one another, where the enriched entrainer fluid is recovered as bottoms product 34 from extractive column 26, and the enriched either 1-chloro-1,1-difluoroethane (HCFC-142b) or 1,1,2-trifluoroethane (HFC-143)) is recovered as in the distillate 38. The recovered enriched entrainer fluid may be recycled and used as entrainer fluid 28 in the extractive column 26, as described previously.

### Examples

### Example 1: Measurement and Characterization of Azeotrope and Azeotrope-Like Compositions of 1,1,2-trifluoroethane (HFC-143) and 1-chloro-1,1-difluoroethane (HCFC-142b)

A set of volume calibrated PTx cells were used to measure azeotrope and azeotrope-like compositions of 1,1,2-trifluoroethane (HFC-143) and 1-chloro-1,1-difluoroethane (HCFC-142b). Mixtures of HFC-143 and HCFC-142b were gravimetrically prepared into evacuated PTx cells; two cells were reserved for measuring each pure component. Once prepared, each of up to eight cells of differing compositions were inserted into a thermostated chamber. In the chamber, each cell was attached to an instrumentation manifold equipped with calibrated pressure transducers and resistance temperature detectors (RTD); this provided a means to measure and record the total saturation pressure of each cell's contents at its local temperature.

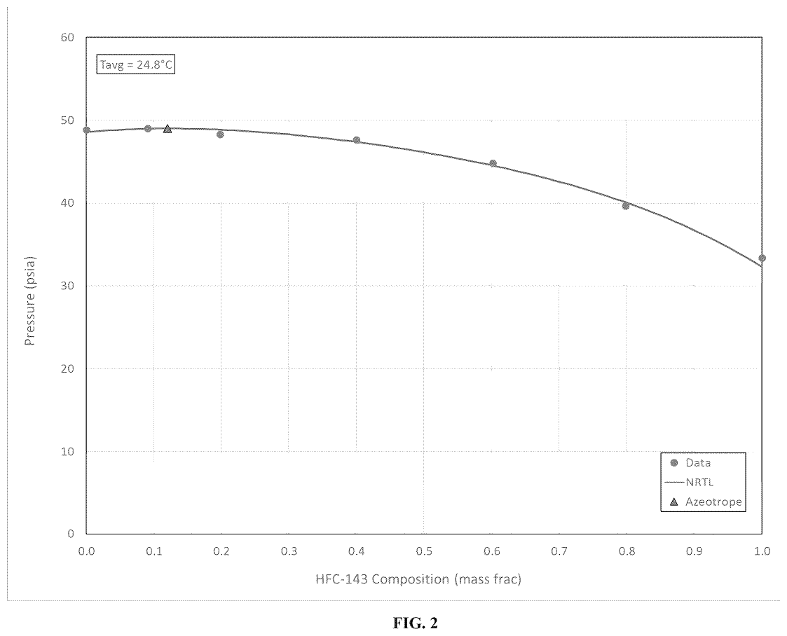

To establish equilibrium at a target temperature, the set point of the chamber was adjusted, yielding an average temperature (T_{avg}) of 24.8° C. across all of the PTx cells. Once at equilibrium, recognized as when temperature and pressures of each cell remain stable for several hours, the local temperature and saturation pressures of each cell were recorded. From these pressure-temperature-composition data, the binary interaction parameters of HFC-143 and HCFC-142b for the Non-random, Two-Liquid (NRTL) were identified. As indicated by the maximum pressure shown in FIG. 2, a minimum boiling azeotrope composition of about 12.2 wt. % HFC-143 and about 87.8 wt. % HCFC-142b was formed based on the data presented in Table 2.

TABLE 2
PTx Study of HFC-143 and HCFC-142b at an average temperature
of 24.8° C. Maximum pressure observed at 9.0 wt.
% HFC-143, azeotrope formed at about 12.2 wt. % HFC-143.

| HFC-143 | HCFC-142b | |
|---|---|---|
| Composition | Composition | Pressure |
| (wt. %) | (wt. %) | (psia) |
| 100.0% | 0.0% | 33.38 |
| 79.8% | 20.2% | 39.66 |
| 60.1% | 39.9% | 44.84 |
| 39.9% | 60.1% | 47.63 |
| 19.7% | 80.3% | 48.31 |
| 9.0% | 91.0% | 49.00 |
| 0.0% | 100.0% | 48.86 |

### Example 2: Azeotrope Locus

The procedure of Example 1 was repeated for each of the temperatures indicated in Table 3 below to generate the azeotropic and azeotrope-like compositional ranges.

Table 3 below includes the azeotrope and azeotrope-like compositions for the HFC-143 and HCFC-142b:

TABLE 3
Azeotrope Locus

| | | | | (2) Azeotropic-Like Composition Range | | | | | |
|---|---|---|---|---|---|---|---|---|---|
| | | (1) Azeotropic | | (i) | | (ii) | | (iii) | |
| | | Composition | | Glide <0.5° C. | | Rel. Vol <1.1 | | Rel. Vol <1.05 | |
| | Saturation | Wt. % | Wt. % | Wt. % | Wt. % | Wt. % | Wt. % | Wt. % | Wt. % |
| Pressure | Temp. | HFC- | HCFC- | HFC- | HCFC- | HFC- | HCFC- | HFC- | HCFC- |
| (psia) | (° C.) | 143 | 142b | 143 | 142b | 143 | 142b | 143 | 142b |
| 21.1 | 0.1 | 6.2 | 93.8 | 0.1 to | 71.5 to | 2.2 to | 81.4 to | 6.0 to | 85.3 to |
| | | | | 28.5 | 99.9 | 18.6 | 97.8 | 14.7 | 94.0 |
| 30.0 | 9.8 | 8.6 | 91.4 | 0.1 to | 68.9 to | 3.6 to | 78.8 to | 8.0 to | 82.9 to |
| | | | | 31.1 | 99.9 | 21.2 | 96.4 | 17.1 | 92.0 |
| 41.9 | 19.8 | 11.0 | 89.0 | 0.1 to | 66.1 to | 4.8 to | 76.2 to | 9.6 to | 80.7 to |
| | | | | 33.9 | 99.9 | 23.8 | 95.2 | 19.3 | 90.4 |
| 49.0 | 24.8 | 12.2 | 87.8 | 0.1 to | 64.7 to | 5.3 to | 74.9 to | 10.3 to | 79.6 to |
| | | | | 35.3 | 99.9 | 25.1 | 94.7 | 20.4 | 89.7 |
| 57.0 | 29.7 | 13.4 | 86.6 | 0.1 to | 63.3 to | 5.8 to | 73.6 to | 11.0 to | 78.5 to |
| | | | | 36.7 | 99.9 | 26.4 | 94.2 | 21.5 | 89.0 |
| 66.0 | 34.7 | 14.5 | 85.5 | 0.1 to | 61.8 to | 6.2 to | 72.4 to | 11.7 to | 77.4 to |
| | | | | 38.2 | 99.9 | 27.6 | 93.8 | 22.6 | 88.3 |
| 75.9 | 39.6 | 15.7 | 84.3 | 0.1 to | 60.1 to | 6.6 to | 71.1 to | 12.3 to | 76.3 to |
| | | | | 39.8 | 99.9 | 28.9 | 93.4 | 23.7 | 87.7 |
| 87.1 | 44.6 | 16.8 | 83.2 | 0.1 to | 58.7 to | 6.9 to | 69.8 to | 12.8 to | 75.3 to |
| | | | | 41.3 | 99.9 | 30.2 | 93.1 | 24.7 | 87.2 |
| 99.2 | 49.5 | 17.9 | 82.1 | 0.1 to | 57.1 to | 7.1 to | 68.5 to | 13.3 to | 74.2 to |
| | | | | 42.9 | 99.9 | 31.5 | 92.9 | 25.8 | 86.7 |
| 113.2 | 54.6 | 19.0 | 81.0 | 0.1 to | 55.3 to | 7.3 to | 67.1 to | 13.9 to | 73.1 to |
| | | | | 44.7 | 99.9 | 32.9 | 92.7 | 26.9 | 86.1 |
| 127.8 | 59.5 | 20.1 | 79.9 | 0.1 to | 53.7 to | 7.4 to | 65.7 to | 14.3 to | 72.0 to |
| | | | | 46.3 | 99.9 | 34.3 | 92.6 | 28.0 | 85.7 |
| 143.7 | 64.4 | 21.1 | 78.9 | 0.1 to | 51.9 to | 7.5 to | 64.3 to | 14.7 to | 70.9 to |
| | | | | 48.1 | 99.9 | 35.7 | 92.5 | 29.1 | 85.3 |
| 161.4 | 69.3 | 22.0 | 78.0 | 0.1 to | 50.1 to | 7.4 to | 62.8 to | 15.0 to | 69.8 to |
| | | | | 49.9 | 99.9 | 37.2 | 92.6 | 30.2 | 85.0 |
| 180.9 | 74.3 | 23.0 | 77.0 | 0.1 to | 48.1 to | 7.3 to | 61.3 to | 15.4 to | 68.6 to |
| | | | | 51.9 | 99.9 | 38.7 | 92.7 | 31.4 | 84.6 |
| 202.1 | 79.3 | 23.9 | 76.1 | 0.1 to | 46.0 to | 7.0 to | 59.7 to | 15.6 to | 67.4 to |
| | | | | 54.0 | 99.9 | 40.3 | 93.0 | 32.6 | 84.4 |

In view of the above data, temperature glide and relative volatility were applied to determine the azeotrope and azeotrope-like compositions.

The temperature glide and relative volatility of a mixture may be derived from thermodynamic measurements, such as those collected via PTx, subject to material balance and thermodynamic constraints. Several methods for deriving temperature glide from thermodynamic measurements are described in Sandler, S. I. (2006). Chapter 10: Vapor-Liquid Equilibrium in Mixtures. In Chemical, Biochemical, and Engineering Thermodynamics (4th ed., pp. 489-574) which includes constraining thermodynamic consistency through the fundamental Gibbs-Duhem relationship and resolving the vapor phase composition, from the measurements, through combined mass balance and equilibrium criteria (frequently referred to as the Rachford-Rice equation or algorithm). Through this derivation, the relationship between equilibrium compositions, temperatures, and pressures are established permitting temperature glide and relative volatility to be evaluated.

For a given composition, the temperature glide, by definition, is the difference between the saturated vapor temperature and the saturated liquid temperature at a fixed pressure in thermodynamic equilibrium. Consequently, an azeotrope composition has a temperature glide of zero and an azeotrope-like composition has a temperature glide that is substantially close to zero. It has been identified that a temperature glide less than 0.5° C. is substantially close to zero and therefore compositions that satisfy such temperature glide are considered azeotrope-like. This is the broad azeotrope-like range.

The relative volatility, by definition, is the ratio of the vapor composition to the liquid composition of the most volatile component relative to the ratio of the vapor composition to the liquid composition of the less volatile component at a fixed pressure in thermodynamic equilibrium. Consequently, an azeotrope composition has a relative volatility of 1.0 and an azeotrope-like composition has a relative volatility that is substantially close to 1.0. It has been identified that a relative volatility of 1.1 is substantially close to 1.0 and therefore compositions that satisfy such relative volatility are considered azeotrope-like. This is the intermediate azeotrope-like range.

Additionally, it has been identified that a relative volatility of 1.05 is substantially close to 1.0 and therefore compositions that satisfy such relative volatility are considered azeotrope-like. This is the narrow azeotrope-like range.

### Example 3: Pressure Swing Separation

A well-known consequence of azeotropic mixtures is the inability to fully separate its constituents in a single continuous distillation operation. For example, separation of a 50/50 mass % mixture of HFC-143 and HCFC-142b by a continuous distillation column held at 24.8 psia, exhibiting azeotropic behavior as described by Example 1, would be bounded by compositions between the HFC-142b endpoint and the azeotropic composition. In other words, distillation of a mixture at these conditions would be unable to produce HCFC-142b in a purity greater than 87.8 mass %. To address this fundamental barrier of azeotropes and attain both purer HFC-143 and HCFC-142b, a different separation strategy must be realized.

As noted by Example 1, the azeotropic composition of HFC-143 and HCFC-142b is sensitive to pressure. This sensitivity can be exploited to support better separation through pressure swing distillation. In this system, a pressure-sensitive azeotrope is separated using two distillation columns in sequence, one at an arbitrary, relatively lower pressure and one at an arbitrary, relatively higher pressure. The columns may be disposed such that the lower pressure column is first in the sequence. Alternatively, the higher-pressure column may be first in the sequence. For the purposes of this example, with reference to FIG. 3, the columns are disposed with the lower pressure column first in the sequence.

A mixture of HFC-143 and HCFC-142b is first subjected to distillation at a lower pressure. The particular composition of the mixture may be tailored as needed. For the purposes of this representative example, a mixture comprising 15 mass % HFC-143 and 85 mass % HCFC-142b is used. Referring to FIG. 3, this mixture, stream 10, is fed to a distillation column 12 at an arbitrary low-pressure.

The feed composition of stream 10 has not yet reached the azeotrope point for the column pressure. As such, the mixture may be separated into fractions enriched in one component of the mixture and the azeotrope or azeotrope-like composition. Here, fractions enriched in the higher boiling point component, HFC-143, are collected as the bottoms shown as stream 16 in FIG. 3. The azeotrope or azeotrope-like composition is the distillate from the low-pressure column 12 shown in FIG. 3. This mixture is then passed to a column 18 at an arbitrary higher pressure, following stream 14 in FIG. 3.

When the composition of stream 14 is brought to the higher pressure of column 18, its composition relative to the higher-pressure azeotropic composition is now lower. This permits fractions enriched in the other component of the mixture to be collected. In this Example, fractions enriched in HCFC-142b are collected as the bottoms product shown as stream 20 in FIG. 3. As with the lower pressure column, the distillate comprises the azeotrope or azeotrope-like mixture. This mixture may be recycled back to co-mingle with low pressure column feed following stream 22 in FIG. 3.

In this way, the barrier of the azeotrope is addressed, using the sensitivity of its composition to the column conditions, to produce two streams each enriched in one of the components. It is important to note that the details in this example are meant to be illustrative. Depending on the context of the mixture, the column conditions and configurations can be designed to support nearly any desired purities of HFC-143 and/or HCFC-142b.

### Example 4: Extractive Distillation

An Azeotrope or azeotrope-like composition of 1,1,2-trifluoroethane (HFC-143) and 1-chloro-1,1-difluoroethane (HCFC-142b) is separated by extractive distillation. Initially, a stream containing an azeotrope or azeotrope-like composition of 1,1,2-trifluoroethane (HFC-143) and 1-chloro-1,1-difluoroethane (HCFC-142b) is fed to a first distillation column, along with an entrainer fluid. The extraction column is operated at a suitable temperature and pressure. The first distillation column acts as an extraction column, where the entrainer fluid and one of the components of the azeotrope or azeotrope-like composition (e.g., component A) is recovered in the bottoms product from the extraction column, and the other component of the azeotrope or azeotrope-like composition (e.g., component B). Here, the entrainer fluid is selected based upon various thermodynamic properties which includes an affinity difference between component A to dissolve in the entrainer fluid vs. component B, where substantially all of the component A is dissolved in the entrainer fluid, while little to none of the component B is dissolved in the entrainer fluid. The column is operated based upon thermodynamic differences between entrainer/component A and the component B, similar to those described in relation to Example 3: pressure swing distillation. The extraction column is operated such that distillate comprises substantially all of the component B, and the bottoms product comprises substantially all of the entrainer fluid and dissolved component A.

The recovered entrainer fluid and dissolved component A is fed to a second recovery column operated at a suitable temperature and pressure. The recovery column separates the entrainer fluid from the component A, as based upon thermodynamic differences, similar to those described in relation to Example 3: pressure swing distillation. Here, substantially all of the entrainer fluid is recovered in the bottoms product from the recovery column, and substantially all of the component A is recovered in the distillate. The recovered entrainer fluid is thereafter recycled to the extraction column.

### Example 5: Process to Separate 1,1,2-trifluoroethane (HFC-143) from 1-chloro-1,1-difluoroethane (HCFC-142b) in a process for manufacturing 1,1,2-trifluoroethane (HFC-143)

A reactant stream including CFCl2-CF2Cl (CFC-113) and hydrogen (H2) is provided to a hydrogenation reactor. The hydrogenation reactor is operated at a temperature between 200° C. to 300° C., at a pressure between 10 psig to 200 psig, and for a contact time between 1 second to 60 seconds. The hydrogenation reactor contains a catalyst comprising platinum on a carbon support. After reacting, the product stream from the reaction is analyzed by the PTx method, similar to Examples 1 through 4 above. The product stream is found to contain at least HCl and an azeotrope or azeotrope-like composition of 1-chloro-1,1-difluoroethane (HCFC-142b) and 1,1,2-trifluoroethane (HFC-143). Specifically, the azeotrope or azeotrope-like composition of 1-chloro-1,1-difluoroethane (HCFC-142b) and 1,1,2-trifluoroethane (HFC-143) is found to contain about 46.0 wt. % to about 99.9 wt. % of 1-chloro-1,1-difluoroethane (HCFC-142b) and from about 0.1 wt. % to about 54.0 wt. % of 1,1,2-trifluoroethane (HFC-143). The product stream is subsequently separated to “break” the azeotrope or azeotrope-like composition of 1-chloro-1,1-difluoroethane (HCFC-142b) and 1,1,2-trifluoroethane (HFC-143) to individual components, where the 1-chloro-1,1-difluoroethane (HCFC-142b) and the 1,1,2-trifluoroethane (HFC-143) are further separated from one another. For example, the azeotrope or azeotrope-like composition of 1-chloro-1,1-difluoroethane (HCFC-142b) and 1,1,2-trifluoroethane (HFC-143) is “broken” and separated by either pressure swing distillation or extractive distillation, as described with reference to Examples 3 and 4 above. 1-chloro-1,1-difluoroethane (HCFC-142b) is removed during the separation process, and the resulting product stream comprises the HCl and 1,1,2-trifluoroethane (HFC-143). The HCl is thereafter removed. The final product recover is found to contain between 90 mol % and 99.5 mol % of 1,1,2-trifluoroethane (HFC-143).

### Aspects

Aspect 1 is an azeotrope or azeotrope-like composition consisting essentially of effective amounts of 1-chloro-1,1-difluoroethane (HCFC-142b) and 1,1,2-trifluoroethane (HFC-143).

Aspect 2 is an azeotrope or azeotrope-like composition according to any preceding or subsequent Aspect, consisting essentially of from about 0.1 wt. % to about 54.0 wt. % of 1,1,2-trifluoroethane (HFC-143) and from about 46.0 to about 99.9 wt. % of 1-chloro-1,1-difluoroethane (HCFC-142b).

Aspect 3 is an azeotrope or azeotrope-like composition according to any preceding or subsequent Aspect, consisting essentially of from about 7.0 wt. % to about 40.3 wt. % of 1,1,2-trifluoroethane (HFC-143) and from about 59.7 to about 93.0 wt. % of 1-chloro-1,1-difluoroethane (HCFC-142b).

Aspect 4 is an azeotrope or azeotrope-like composition according to any preceding or subsequent Aspect, consisting essentially of from about 15.6 wt. % to about 32.6 wt. % of 1,1,2-trifluoroethane (HFC-143) and from about 67.4 to about 84.4 wt. % of 1-chloro-1,1-difluoroethane (HCFC-142b).

Aspect 5 is an azeotrope or azeotrope-like composition according to any preceding or subsequent Aspect, consisting essentially of about 23.9 wt. % of 1,1,2-trifluoroethane (HFC-143) and of about 76.1 wt. % of 1-chloro-1,1-difluoroethane (HCFC-142b).

Aspect 6 is an azeotrope or azeotrope-like composition according to any preceding or subsequent Aspect, wherein the azeotrope or azeotrope-like composition has a boiling point of from about 0.1° C. at a pressure of about 21.1 psia, to about 79.3° C. at a pressure of about 202.1 psia.

Aspect 7 is a method for producing 1,1,2-trifluoroethane (HFC-143) comprising: hydrogenating 1,1,2-trichloro-1,2,2-trifluoroethane (CFC-113) with hydrogen (H2) to form a product mixture, the product mixture comprising an azeotrope or azeotrope-like composition consisting essentially of effective amounts of 1-chloro-1,1-difluoroethane (HCFC-142b) and 1,1,2-trifluoroethane (HFC-143); and separating the 1-chloro-1,1-difluoroethane (HCFC-142b) and the 1,1,2-trifluoroethane (HFC-143) to provide a product composition comprising the 1,1,2-trifluoroethane (HFC-143).

Aspect 8 is a method accordingly to any proceeding or subsequent Aspect, wherein the separating step comprises: conveying the product mixture to a first column having a first pressure; collecting a first bottoms product from the first column; conveying a first distillate from the first column to a second column having a second pressure to provide a second distillate and a second bottoms product, the second distillate comprising the azeotrope or azeotrope-like composition; and collecting a second bottoms product from the second column.

Aspect 9 is a method according to any proceeding or subsequent Aspect, wherein the first column pressure is lower than the second column pressure, the first bottoms product consists essentially of 1,1,2-trifluoroethane (HFC-143) and the second bottoms product consists essentially of 1-chloro-1,1-difluoroethane (HCFC-142b).

Aspect 10 is a method according to any proceeding or subsequent Aspect, wherein the first column pressure is higher than the second column pressure, the first bottoms product consists essentially of 1,1,2-trichloro-1,2,2-trifluoroethane (CFC-113), and the second bottoms product consists essentially of 1-chloro-1,1-difluoroethane (HCFC-142b).

Aspect 11 is a method according to any proceeding or subsequent Aspect, comprising the additional step of recycling the second distillate to the first column.

Aspect 12 is a method according to any proceeding or subsequent Aspect, wherein the separating step comprises: conveying the product mixture and an entrainer fluid to a first column; collecting a first distillate from the first column comprising a first component of the azeotrope or azeotrope-like composition; collecting a first bottoms product from the first column comprising a mixture of the entrainer and a second component of the azeotrope or azeotrope-like composition; conveying the first bottoms product to a second column to separate the entrainer and the second component of the azeotrope or azeotrope-like composition; and removing a composition comprising of the second component of the azeotrope or azeotrope-like composition as a second distillate from the second column.

Aspect 13 is a method according to any proceeding or subsequent Aspect, wherein the first component of the azeotrope or azeotrope-like composition consisting essentially of 1-chloro-1,1-difluoroethane (HCFC-142b), and the second component of the azeotrope or azeotrope-like composition consisting essentially of 1,1,2-trifluoroethane (HFC-143).

Aspect 14 is a method according to any proceeding or subsequent Aspect, wherein the first component of the azeotrope or azeotrope-like composition consisting essentially of 1-chloro-1,1-difluoroethane (HCFC-142b), and the second component of the azeotrope or azeotrope-like composition consisting essentially of 1,1,2-trichloro-1,2,2-trifluoroethane (CFC-113).

Aspect 15 is a method according to any proceeding or subsequent Aspect, further comprising the additional step of recycling a second bottoms product consisting essentially of the entrainer from the second column to the first column.

Aspect 16 is an azeotrope or azeotrope-like composition produced by the method of any one of Aspects 7 through 15.

Aspect 17 is a composition comprising an azeotrope or azeotrope-like composition consisting essentially of any of Aspects 1-6 or resulting from any of Aspects 7-16.

Aspect 18 is a composition comprising 1,1,2-trifluoroethane (HFC-143), produced by the method of any of Aspects 7-15.

What is claimed is:

1. An azeotrope or azeotrope-like composition consisting essentially of effective amounts of 1-chloro-1,1-difluoroethane (HCFC-142b) and 1,1,2-trifluoroethane (HFC-143).

2. The azeotrope or azeotrope-like composition of claim 1, consisting essentially of from about 0.1 wt. % to about 54.0 wt. % of 1,1,2-trifluoroethane (HFC-143) and from about 46.0 to about 99.9 wt. % of 1-chloro-1,1-difluoroethane (HCFC-142b).

3. The azeotrope or azeotrope-like composition of claim 1, consisting essentially of from about 7.0 wt. % to about 40.3 wt. % of 1,1,2-trifluoroethane (HFC-143) and from about 59.7 to about 93.0 wt. % of 1-chloro-1,1-difluoroethane (HCFC-142b).

4. The azeotrope or azeotrope-like composition of claim 1, consisting essentially of from about 15.6 wt. % to about 32.6 wt. % of 1,1,2-trifluoroethane (HFC-143) and from about 67.4 to about 84.4 wt. % of 1-chloro-1,1-difluoroethane (HCFC-142b).

5. The azeotrope or azeotrope-like composition of claim 1, consisting essentially of about 23.9 wt. % of 1,1,2-trifluoroethane (HFC-143) and about 76.1 wt. % of 1-chloro-1,1-difluoroethane (HCFC-142b).

6. The azeotrope or azeotrope-like composition of claim 1, wherein the azeotrope or azeotrope-like composition has a boiling point of from about 0.1° C. at a pressure of about 21.1 psia, to about 79.3° C. at a pressure of about 202.1 psia.

7. A method for producing 1,1,2-trifluoroethane (HFC-143) comprising:
hydrogenating 1,1,2-trichloro-1,2,2-trifluoroethane (CFC-113) with hydrogen (H2) to form a product mixture, the product mixture comprising an azeotrope or azeotrope-like composition consisting essentially of effective amounts of 1-chloro-1,1-difluoroethane (HCFC-142b) and 1,1,2-trifluoroethane (HFC-143); and
separating the 1-chloro-1,1-difluoroethane (HCFC-142b) and the 1,1,2-trifluoroethane (HFC-143) to provide a product composition comprising the 1,1,2-trifluoroethane (HFC-143).

8. The method of claim 7, wherein the separating step comprises:
conveying the product mixture to a first column having a first pressure;
collecting a first bottoms product from the first column;
conveying a first distillate from the first column to a second column having a second pressure to provide a second distillate and a second bottoms product, the second distillate comprising the azeotrope or azeotrope-like composition; and
collecting a second bottoms product from the second column.

9. The method of claim 8, wherein the first column pressure is lower than the second column pressure, the first bottoms product consists essentially of 1,1,2-trifluoroethane (HFC-143) and the second bottoms product consists essentially of 1-chloro-1,1-difluoroethane (HCFC-142b).

10. The method of claim 8, wherein the first column pressure is higher than the second column pressure, the first bottoms product consists essentially of 1,1,2-trifluoroethane (HFC-143), and the second bottoms product consists essentially of 1-chloro-1,1-difluoroethane (HCFC-142b).

11. The method of claim 8, comprising the additional step of recycling the second distillate to the first column.

12. The method of claim 7, wherein the separating step comprises:
conveying the product mixture and an entrainer fluid to a first column;
collecting a first distillate from the first column comprising a first component of the azeotrope or azeotrope-like composition;
collecting a first bottoms product from the first column comprising a mixture of the entrainer and a second component of the azeotrope or azeotrope-like composition;
conveying the first bottoms product to a second column to separate the entrainer and the second component of the azeotrope or azeotrope-like composition; and
removing a composition comprising the second component of the azeotrope or azeotrope-like composition as a second distillate from the second column.

13. The method of claim 12, wherein the first component of the azeotrope or azeotrope-like composition consisting essentially of 1,1,2-trifluoroethane (HFC-143), and the second component of the azeotrope or azeotrope-like composition consisting essentially of 1-chloro-1,1-difluoroethane (HCFC-142b).

14. The method of claim 12, wherein the first component of the azeotrope or azeotrope-like composition consisting essentially of 1-chloro-1,1-difluoroethane (HCFC-142b), and the second component of the azeotrope or azeotrope-like composition consisting essentially of 1,1,2-trifluoroethane (HFC-143).

15. The method of claim 12, further comprising the additional step of recycling a second bottoms product consisting essentially of the entrainer from the second column to the first column.
